(19) United States
(12) Patent Application Publication
(10) Pub. No.: US 2004/0158066 A1
(43) Pub. Date: Aug. 12, 2004

(54) Novel pyridylmethylaminopyrimidines

(76) Inventors: Vittoria Chiesa, Constance (DE); Gerhard Grundler, Constance (DE)

Correspondence Address: Nath & Associates, Sixth Floor, 1030 15th Street NW, Washington, DC, 20005, US

(21) Appl. No.: 10/477,621

(22) PCT Filed: May 14, 2002
(86) PCT No.: PCT/EP02/05265

(30) Foreign Application Priority Data
May 18, 2001 (EP) 011122225.6

(51) Int. Cl. C 07D 4 3/14

(52) U.S. Cl. 544/328

(57) ABSTRACT

Pyridylmethylaminopyrimidine compounds of a certain general formula I, in which the substituents and symbols are as defined in the description, are suitable for controlling Helicobacter bacteria.

### FIELD OF THE INVENTION

The invention relates to compounds intended for use in the pharmaceutical industry as active principles for preparing medicaments.

### STATE OF THE ART

International patent application WO 96/16656 describes compounds of a general formula A—X—R in which A may be a fused imidazolyl radical and R may be a nonaromatic hydrocarbon radical. European patent application EP 632040 describes further fused imidazoles which carry as substituent B a 5- or 6-membered fused or nonfused unsubstituted heterocycle. International patent application WO 98/28299 describes imidazopyridazines attached via a specific bridge in position 4 to a pyridine ring substituted in position 2. International patent application WO 99/61439 describes pyridylmethylaminopyrimidines substituted in a special way in position 4. All of the compounds specified in the above documents are said to be suitable for controlling Helicobacter bacteria.

### DESCRIPTION OF THE INVENTION

The invention provides compounds of the formula I

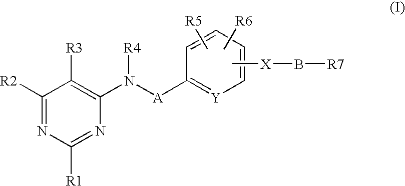

in which

R1 is hydrogen, 1-4C-alkyl or halogen,

R2 is hydrogen, 1-4C-alkyl or halogen,

R3 is hydrogen, 1-4C-alkyl or halogen,

R4 is hydrogen or 1-4C-alkyl,

R5 is hydrogen, hydroxyl, 1-4C-alkyl, 1-4C-alkoxy, wholly or predominantly fluorine-substituted 1-4C-alkoxy, trifluoromethyl or halogen,

R6 is hydrogen, hydroxyl, 1-4C-alkyl, 1-4C-alkoxy, wholly or predominantly fluorine-substituted 1-4C-alkoxy or halogen,

R7 is a cydic or bicyclic radical which is substituted by nitro and R8 and R9 and is selected from the group consisting of imidazole, imidazopyridazine and imidazopyridine,

A is 1 -7C-alkylene,

B is a bond or 1-7C-alkylene,

X is O (oxygen), N-1-4C-alkyl, NH or S(O)ₙ and

Y is N,

where

R8 is hydrogen, 1-4C-alkyl, halogen, nitro, hydroxy-1 -4C-alkyl or 1-4C-alkylcarbonyloxy-1-4C-alkyl,

R9 is hydrogen, 1-4C-alkyl or nitro, and

n is 0, 1 or 2,

and salts thereof.

1-4C-Alkyl stands for straight-chain, branched or cyclic alkyl radicals having from 1 to 4 carbon atoms. Examples that may be mentioned include the butyl, isobutyl, sec-butyl, tert-butyl, cyclobutyl, propyl, isopropyl, cylopropyl, cyclopropylmethyl, ethyl, and methyl radicals.

Halogen for the purposes of the present invention is bromine, chlorine, and fluorine.

1-4C-Alkoxy stands for a radical which in addition to the oxygen atom contains one of the abovementioned 1-4C-alkyl radicals. Examples that may be mentioned include the cyclopropylmethoxy, methoxy, and ethoxy radicals.

Wholly or predominantly fluorine-substituted 1-4C-alkoxy stands for a 1-4C-alkoxy radical in which all or more than half of the hydrogen atoms have been replaced by fluorine atoms. Examples that may be mentioned include the 2,2,3,3,3-pentafluoropropoxy, the perfluoroethoxy, the 1,2,2-trifluoroethoxy, particularly the 1,1,2,2-tetrafluoroethoxy, the 2,2,2-trifluoroethoxy, the trifluoromethoxy, and, in particular, the difluoromethoxy radicals.

1-7C-Alkylene stands for straight-chain or branched 1-7C-alkylene radicals, examples being the methylene(—CH₂—), ethylene(—CH₂—CH₂—), trimethylene(—CH₂—CH₂—CH₂—), tetramethylene(—CH₂—CH₂—CH₂—CH₂—), 1,2-dimethylethylene[—CH(CH₃)—CH(CH₃)—], 1,1-dimethylethylene[—C(CH₃)₂—CH₂—], 2,2-dimethylethylene[—CH₂—C(CH₃)2—], isopropylidene[—C(CH₃)₂—], 1-methylethylene[—CH(CH₃)—CH₂—], pentamethylene (—CH₂—CH₂—CH₂—CH₂—CH₂—), hexamethylene(—CH₂—CH₂—CH₂—CH₂—CH₂—CH₂—), and the heptamethylene(—CH₂—CH₂—CH₂—CH₂—CH₂—CH₂—CH₂—)radicals.

Hydroxy-1-4C-alkyl stands for the abovementioned 1-4C-alkyl radicals substituted by a hydroxyl group. Examples that may be mentioned include the 2-hydroxyethyl and 3-hydroxypropyl radicals and, in particular, the hydroxymethyl radical.

1-4C-Alkylcarbonyloxy radicals contain in addition to the oxygen atom one of the abovementioned 1-4C-alkylcarbonyl radicals. An example that may be mentioned is the acetoxy radical (CH₃CO—O—).

1-4C-Alkylcarbonyloxy-1-4C-alkyl stands for one of the abovementioned 1-4C-alkyl radicals substituted by one of the abovementioned 1-4C-alkylcarbonyloxy radicals. An example that may be mentioned is the acetoxymethyl (CH₃CO—O—CH₂—) radical.

As exemplary radicals R7 mention may be made of the 2-methyl-5-nitroimidazol-1-yl radical, the 2-methyl-4-nitroimidazol-1-yl radical, the 5-bromo-2-methyl-4-nitroimidazol-1 -yl radical, the 4-nitrolmidazol-1-yl radical, the 2-methyl-4,5-dinitroimidazol-1-yl radical, the 2,4-dinitroimidazol-1-yl radical, the 2-hydroxymethyl-5-nitroimidazol-1-yl radical, the 2-acetoxymethyl-5-nitroimidazol-1-yl radical, the 3-nitroimidazo[1,2-a]pyridin-8-yl radical, the 2-methyl-3-nitroimidazo[1,2-a]pyridin-8-yl radical, the 3-nitroimidazo[1,2-a]pyridin-6-yl radical, the 3-nitroimidazo[1,2-b]pyridazin-7-yl radical, and the 3-nitroimidazo[1,2-b]pyridazin-6-yl radical.

Suitable salts for compounds of the formula I depending on substitution, include all acid addition salts or all salts with bases. Particular mention may be made of the pharmacologically acceptable salts of the organic and inorganic acids and bases that are commonly used in pharmacy. Suitable salts of this kind include, on the one hand, water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, the acids being used in an equimolar proportion or in a proportion which deviates from equimolarity for preparing the salts, depending on whether the acid in question is monobasic or polybasic and on the particular salt desired.

On the other hand, salts with bases are also suitable. Examples of salts with bases that may be mentioned include alkali metal (lithium, sodium, potassium) or calcium, aluminum, magnesium, titanium, ammonium, meglumine or guanidinium salts, salt preparation here too being carried out using the bases in an equimolar proportion or in a proportion which deviates from equimolarity.

Pharmacologically unacceptable salts, which may be initially obtained, for example, during the preparation of the compounds of the invention on the industrial scale as process products, are converted into pharmacologically acceptable salts by methods known to the skilled worker.

The skilled worker is aware that the compounds of the invention and their salts, if isolated for example in crystalline form, may contain various amounts of solvents. The invention therefore further embraces all solvates and in particular all hydrates of the compounds of the formula I, and also all solvates and in particular all hydrates of the salts of the compounds of the formula I.

Compounds according to the invention which are to be emphasized are those of the formula I in which

R1 is hydrogen, 1-4C-alkyl or halogen,

R2 is hydrogen, 1-4C-alkyl or halogen,

R3 is hydrogen or halogen,

R4 is hydrogen or 1-4C-alkyl,

R5 is hydrogen, hydroxyl, 1-4C-alkyl, 1-4C-alkoxy, wholly or predominantly fluorine-substituted 1-4C-alkoxy, trifluoromethyl or halogen,

R6 is hydrogen, 1-4C-alkyl, 1-4C-alkoxy or halogen,

R7 is a cyclic or bicyclic radical which is substituted by nitro and R8 and R9 and is selected from the group consisting of imidazole and imidazopyridine,

A is methylene,

B is a bond or 1-4C-alkylene,

X is O (oxygen), NH or S(O)ₙ and

Y is N,

where

R8 is hydrogen,

R9 is hydrogen,

n is 0,

and salts thereof.

One embodiment of the compounds deserving of emphasis (embodiment a) are those of the formula I in which B is a bond and R7 is an imidazopyridazine radical substituted by nitro and the radicals R8 and R9.

A further embodiment of the compounds deserving of emphasis (embodiment b) are those of the formula I in which B is an ethylene radical and R7 is an imidazole radical substituted by nitro and the radicals R8 and R9.

Compounds of the invention deserving of particular emphasis are those of the formula I in which

R1 is hydrogen or methyl,

R2 is hydrogen or methyl,

R3 is hydrogen or chlorine,

R4 is hydrogen or methyl,

R5 is hydrogen, 1-4C-alkyl, 1-4C-alkoxy, wholly or predominantly fluorine-substituted 1-4C-alkoxy, trifluoromethyl or halogen,

R6 is hydrogen, 1-4C-alkyl, 1-4C-alkoxy or halogen,

R7 is a 3nitroimidazo[1,2-b]pyridazin-6-yl radical or a 2-methyl-5-nitroimidazol-1-yl radical,

A is methylene,

B is a bond or 1-2C-alkylene,

X is O (oxygen), NH or S, and

Y is N,

and salts thereof.

Preferred compounds of embodiment a are those in formula I*

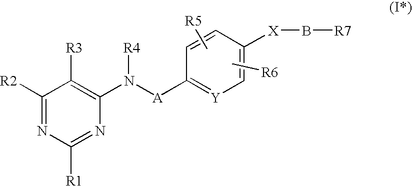

in which

R1 is hydrogen or methyl,

R2 is hydrogen or methyl,

R3 is hydrogen or chlorine,

R4 is hydrogen or methyl,

R5 is hydrogen, hydroxyl, methyl, methoxy, ethoxy, cyclopropylmethoxy, isobutoxy, trifluoromethoxy, difluoromethoxy, trifluoromethyl or chlorine,

R6 is hydrogen, methyl, methoxy or chlorine,

R7 is a 3-nitroimidazo[1,2-b]pyridazin-6-yl radical,

A is methylene,

B is a bond,

X is O (oxygen), NH or S, and

Y is N,

and salts thereof.

Preferred compounds of embodiment b are those in formula I* in which

R1 is hydrogen or methyl,

R2 is hydrogen or methyl,

R3 is hydrogen or chlorine,

R4 is hydrogen or methyl,

R5 is hydrogen, hydroxyl, methyl, methoxy, ethoxy, cyclopropylmethoxy, isobutoxy, trifluoromethoxy, difluoromethoxy, trifluoromethyl or chlorine,

R6 is hydrogen, methyl, methoxy or chlorine,

R7 is a 2-methyl-5nitroimidazo-1-yl radical,

A is methylene,

B is ethylene,

X is O (oxygen), NH or S, and

Y is N,

and salts thereof.

Particularly preferred compounds of embodiment a are those in formula I* in which

R1 is methyl,

R2 is methyl,

R3 is chlorine,

R4 is hydrogen,

R5 is hydrogen or methyl,

R6 is hydrogen or methoxy,

R7 is a 3-nitroimidazo[1,2-b]pyridazin-6-yl radical,

A is methylene,

B is a bond,

X is O (oxygen), and

Y is N,

and salts thereof.

Particularly preferred compounds of embodiment b are those in formula I* in which

R1 is methyl,

R2 is methyl,

R3 is chlorine,

R4 is hydrogen,

R5 is hydrogen or methyl,

R6 is hydrogen or methoxy,

R7 is a 2-methyl-5-nitroimidazo1-yl radical,

A is methylene,

B is ethylene,

X is O (oxygen), and

Y is N,

and salts thereof.

The compounds of the formula I according to the invention may be synthesized in a variety of ways. In principle the compounds of the formula I may be prepared in conventional manner by reacting the compounds of the formula II with the compounds of the formula III (in which L is an eliminable group, e.g., a halogen atom, especially chlorine, or a mesyloxy group).

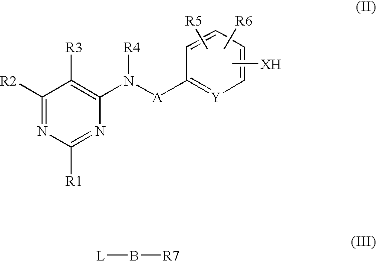

The reaction of the compounds of the formula II with the compounds of the formula III takes place, for example, as described by way of example in the section “Examples”, preferably in inert anhydrous solvents (such as dimethylformamide, for example) in the presence of an organic or, preferably, inorganic auxiliary base (such as potassium carbonate, for example).

The compounds of the formulae II and III are known (see e.g. B. Kohl et al., J. Med. Chem. 1992, 35, 1049-1057; C. Guet et al., J. Chem. Res. Miniprint 1982, 9, 2515-2527; W. M. Galebiewski et al., Bull. Pol. Acad. Sci. Chem. 1990, 38, 17-27; Jen et al. J. Med. Chem. 1977, 20, 1258-1261; D. Scopes et al., J. Med. Chem. 1992, 35, 490-501) or may be prepared as described in the examples below under “Starting compounds” or in analogy thereto from corresponding known compounds in conventional manner using customary process steps.

The examples which follow illustrate the invention without restricting it. The compounds of the invention and the starting compounds may be prepared in a manner analogous to that described in the examples. The abbreviation m.p. denotes melting point, conc. stands for “concentrated”, h stands for hour(s), and min for minute(s). The compounds named as end products and the salts of these compounds are a particularly preferred subject matter of the invention.

The reaction of the compounds of the formula II with the compounds of the formula III takes place, for example, as described by way of example in the section “Examples”, preferably in inert anhydrous solvents (such as dimethylformamide, for example) in the presence of an organic or, preferably, inorganic auxiliary base (such as potassium carbonate, for example).

The compounds of the formulae II and III are known (see e.g. B. Kohl et al., J. Med. Chem. 1992, 35, 1049-1057; C. Guet et al., J. Chem. Res. Miniprint 1982, 9, 2515-2527; W. M. Galebiewski et al., Bull. Pol. Acad. Sci. Chem. 1990, 38,17-27; Jen et al. J. Med. Chem. 1977, 20, 1258-1261; D. Scopes et al., J. Med. Chem. 1992, 35, 490-501) or may be prepared as described in the examples below under “Starting compounds” or in analogy thereto from corresponding known compounds in conventional manner using customary process steps.

The examples which follow illustrate the invention without restricting it. The compounds of the invention and the starting compounds may be prepared in a manner analogous to that described in the examples. The abbreviation m.p. denotes melting point, conc. stands for “concentrated”, h stands for hour(s), and min for minute(s). The compounds named as end products and the salts of these compounds are a particularly preferred subject matter of the invention.

### EXAMPLES

### End Products

### 1. (5-Chloro-2,6-dimethylpyrimidin-4-yl)[4-methoxy-3-methyl-5-(3-nitroimidazo[1,2-b]pyridazin-6-yloxy)pyridin-2-ylmethyl]amine

1.0 g (3.2 mmol) of 6-[(5-chloro-2,6-dimethypyrimidin-4-ylamino)methyl]-3-hydroxy-4-methoxy5-methylpyridine is suspended in 30 ml of anhydrous dimethylformamide and the suspension is heated to 80° C. 2.2 g (16.0 mmol) of potassium carbonate and 0.58 g (2.9 mmol) of 6-chloro-3-nitroimidazo[1,2-b]pyridazine are added and the mixture is stirred at 80° C. for 1 h. After cooling to room temperature, the mixture is poured into water (200 ml) and extracted with methylene chloride/methanol 4:1 (3×200 ml). The organic extracts are dried over sodium sulfate and concentrated. After drying of the residue in a vacuum drying cabinet at 40° C., 0.75 g (55%) of the title compound is isolated as a white powder. m.p. 229.5-231.5° C.

### 2. (5-Chloro-2,6-dimethylpyrimidin-4-yl){4-methoxy-3-methyl-5-[2-(2-methyl -5-nitroimidazol-1-yl)ethoxy]pyridin-2-ylmethyl}amine

2.0 g (6.4 mmol) of 6-[(5-chloro-2,6-dimethyipyrimidin-4-ylamino)methyl]-3-hydroxy-4-methoxy-5-methylpyridine are suspended in 30 ml of anhydrous dimethylformamide and the suspension is heated to 80° C. 4.4 g (32.0 mmol) of potassium carbonate are added and the mixture is stirred for 1 h. Then a solution of 1.69 (6.4mmol) of 2-(2-methyl-5-nitroimidazol-1-yl)ethyl methanesulfonate in 10 ml of dimethylformamide is slowly added dropwise and the mixture is stirred at 80° C. for 1 h. After cooling to room temperature, the mixture is poured into water (200 ml) and the precipitate is filtered off with suction. After drying in a vacuum drying cabinet at 40° C., 1.0 g (33.8%) of the title compound is isolated as a beige powder. m.p. 182.5-184° C.

### 3. (5-Chloro-2,6dimethylpyrimidin-4-yl){5-(3-nitroimidazo[1,2-b]pyridazin-6-yloxy)pyridin-2-ylmethyl}amine

0.5 g (1.9 mmol) of 6-[(5-chloro-2.6-dimethylpyrimidin-4-ylamino)methyl]-3-hydroxypyridine is dissolved in 15 ml of anhydrous dimethylformamide. 1.3 g (9.5 mmol) of potassium carbonate and 0.33 g (1.7 mmol) of 6-chloro-3-nitroimidazo[1,2-b]pyridazine are added. The mixture is stirred at room temperature for 30 min and then heated at 80° C. for 1 h. After cooling to room temperature, the mixture is poured into water (250 ml) and the precipitate is filtered off with suction to give, after drying in a vacuum drying cabinet at 40° C., 0.69 g (95.1%) of the title compound as an orange solid. m.p. 194-198° C.

### 4. (5-Chloro-2,6-dimethylpyrimidin-4-yl){5-[2-(2-methyl-5-nitroimidazol-1-yl)ethoxy]pyridine-2-ylmethyl}amine

1.5 g (4.3 mmol) of 6-[(5-chloro-2,6-dimethylpyrimidin-4-ylamino)methyl]-3-hydroxypyridine are dissolved in 30 ml of anhydrous dimethylformamide at 80° C. 3.0 h (21.5 mmol) of potassium carbonate are added and the mixture is stirred for 30 min. Then a solution of 2.2 g (8.7 mmol) of 2-(2-methyl-5-nitroimidazol-1-yl)ethyl methanesulfonate in 10 ml of dimethylformamide is slowly added dropwise and the mixture is stirred at 80° C. for 1 h. After cooling to room temperature, the mixture is poured into water (200 ml) and the precipitate is filtered off with suction to give, after drying in a vacuum drying cabinet at 40° C., 0.57 g (31.7%) of the title compound as a light-colored powder. m.p. 181.5-183.5° C.

### 5. (5-Chloro-2,6-dimethylpyrimidin-4-yl)-[5-(3-nitroimidazo[1,2-b]pyridazin-6-yloxy)pyridin-2-ylmethyl]amine dihydrochloride

2.0 g (4.7 mmol) of (5-chloro-2,6-dimethylpyrimidin-4-yl)-[6-chloro-5-(3-nitroimidazo[1,2-b]pyridazin-6-yloxy)pyridin-2-ylmethyl]amine are suspended in 500 ml of acetone, and the suspension is heated to reflux. 800 μl (9.4 mmol) of conc. hydrochloric acid are added, and the mixture is stirred at room temperature overnight. Following filtration with suction and drying of the precipitate in a vacuum drying cabinet at 40° C., 2.2 g (94%) of the title compound are isolated as a white solid. M.p. 280°-281° C.

### 6. (5-Chloro-2,6-dimethylpyrimidin-4-yl)-[5-(3-nitroimidazo[1,2-b]pyridazin-6-yloxy)pyridin-2-ylmethyl]amine sulfate

Similarly to the procedure described in example 6, 2.0 g (4.7 mmol) of (5-chloro-2,6-dimethylpyrimidin-4-yl)[6-5-(3-nitroimidazo[1,2-b]pyridazin-6-yloxy)pyridin-2-ylmethyl]amine and 256 μl (4.7 mmol) of sulfuric acid are reacted In 500 ml of acetone. The precipitate is filtered off with suction, giving 2.3 g (95%) of the title compound as a white solid. M.p. 233°-234° C.

### 7. (5-Chloro-2,6-dimethylpyrimidin-4-yl)-[6-chloro-5-(3-nitroimidazo[1,2-b]pyridazin-6-yloxy)pyridin-2-ylmethyl]amine

0.8 g (2.7 mmol) of 2-chloro-6-[(5-chloro-2,6-dimethylpyrimidin-4-ylamino)methy]-3-hydroxypyridine (from example K) is dissolved in 30 ml of anhydrous dimethylformamide. 1.9 g (13.5 mmol) of potassium carbonate are added, and the mixture is stirred at 80° C. for 30 min. 0.67 g (2.7 mmol) of 6-chloro-3-nitroimidazo[1,2-b]pyridazine is then added, and the reaction mixture is heated at 80° C. for 2 h. The mixture is slowly cooled to room temperature and then added to water (100 ml) and extracted with ethyl acetate (3×50 ml). The organic extracts are dried over magnesium sulfate and concentrated. Following crystallization of the crude product from diisopropyl ether, 720 mg (58%) of the title compound are isolated as a pale solid. M.p. 184°-186° C.

### 8. (5-Chloro-2,6-dimethylpyrimidin-4-yl)-{6-chloro-5-[2-(2-methyl-5-nitroimidazol-1-yl)ethoxy]pyridin-2-ylmethyl}amine

0.8 g (2.7 mmol) of 2-chloro-6-[(5-chloro-2,6-dimeththyl pyrimidin-4-ylamino)methyl]-3-hydropyridine (from example K) is suspended in 20 ml of anhydrous dimethylformamide, and the suspension is heated to 80° C. 1.87 g (13.5 mmol) of potassium carbonate are added, and the mixture is stirred for 30 min. A solution of 0.67 g (2.7 mmol) of 2-(2-methyl-4-nitroimidazol-1-yl)ethyl methanesulfonate in 10 ml of anhydrous dimethylformamide is added dropwise, and the reaction mixture is stirred at 80° C. for 3 h. The mixture is cooled to room temperature and then added to water (400 ml), and the precipitate is filtered off with suction. After drying of the precipitate in a vacuum drying cabinet at 40° C., 1.0 g (86%) of the title compound is isolated as a beige solid. M.p. 206°-208° C.

### 9. (5-Chloro-2,6-dimethylpyrimidin-4-yl)methyl-[5-(3-nitroimidazo[1,2-b]pyridazin-6-yloxy)pyridin-2-ylmethyl]amine

0.9 g (3.23 mmol) of 6{[(5-chloro-2,6-dimethylpyrimidin-4-yl)methylamino]methyl}-3-hydroxypyridine (from example L) is dissolved in 10 ml of anhydrous dimethylformamide, and 2.2 g (16.1 mmol) of potassium carbonate are added. The mixture is stirred at 80° C. for 1 h, and 641 mg (3.23 mmol) of 6-chloro-3-nitroimidazo[1,2-b]pyridazine are then added. The reaction mixture is heated at 80° C. for 2 h, slowly cooled to room temperature and then added to water (50 ml). The precipitate is filtered off with suction and dried in a vacuum drying cabinet at 40° C. 2.6 g (79%) of the title compound are isolated as a beige solid. M.p. 183°-184° C.

### 10. [6-Bromo-5-(3-nitro-imidazo[1,2b]pyridazin-6-yloxy)-4-(2,2,2-trifluoroethoxy)-pyridinl-2-ylmethyl]-(5-chloro-2,6-dimethyl-pyrimidin-4-yl)-amine

A solution of 0.43 g (1.0 mmol) of 2-bromo-6-[(5-chloro-2,6-dimethyl-pyrimidin-4-ylamino)-methyl]-3-hydroxy-4-(2,2,2-trifluoroethoxy)-pyridine (from example I) in dry N,N-dimethylformamide (5 ml) is treated with 0.7 g (5.0 mmol) of potassium carbonate and the reaction mixture is stirred at 80° C. for 1 h. A solution of 0.2 g (1.0 mmol) of 6-chloro-3-nitro-imidazo[1,2b]pyridazine in dry N,N-dimethylformamide (2 ml) is added dropwise at 80° C. and the reaction mixture is stirred for 8h. The mixture is cooled to room temperature, poured into water (30 ml) and extracted with ethyl acetate (3×20 ml). The combined organic layers are dried with magnesium sulphate and concentrated in vacuo. After purification of the residue by chromatography on silica gel (petroleum ether/ethyl acetate=1:1) and recrystallization from petroleum ether 15 mg (3%) of the target compound are isolated as a beige powder of m.p. 180°-182° C.

### Starting Compounds

### A. 3-Hydroxy-5-benzyloxy-4-methoxy-5,6-dimethylpyrldine

50 g (0.33 mol) of 3-hydroxy-4-methoxy-5,6-dimethylpyridine are added in portions to a suspension of 13.29 (0.33 mol) of sodium hydride (60% suspension in liquid paraffin) in 150 ml of anhydrous dimethylformamide and the mixture is heated at 60° C. with vigorous stirring for 1 h. A solution of 39.3 ml (0.33 mol) of benzyl bromide in anhydrous dimethylformamide is added dropwise over 1 h. The mixture is stirred at 60° C. for a further 1 h and then cooled to room temperature. The mixture is poured into 500 ml of water and extracted with ethyl acetate (3×200 ml). The organic phases are dried over sodium sulfate and concentrated. The residue (65.0 g) is purified by chromatography on silica gel (petroleum ether/ethyl acetate=2:1+5% ammonia). The fractions containing the product are collected and concentrated. After drying in a vacuum drying cabinet at 40° C., 50.5 g (63.7%) of the title compound are isolated as a white powder. m.p. 152-155° C.

### B. 5-Benzyloxy-4-methoxy-2,3-dimethylpyridine N-oxide

40.0 g (0.22 mol) of 80% m-chloroperoxybenzoic acid are added in portions to a solution of 50.0 g (0.20 mol) of 5-benzyloxy-4-methoxy-5,6-dimethylpyridine in 200 ml of anhydrous dichloromethane. The mixture is cooled to 0° C. for 30 min. It is then warmed to room temperature and stirred for 15 h. 2×5.0 g of m-chloroperoxybenzoic acid are added and the mixture is stirred for a further 2 h and then poured into water. The organic phase is washed with 3×50 ml of saturated sodium bicarbonate solution and extracted with dichloromethane. The organic extracts are dried over sodium sulfate and concentrated. This gives 50.0 g (93.6%) of the title compound as a yellow oil [NMR: δ=2.15 (s, 3H, Me), 2.22 (s, 3H, Me), 3.81 (s, 3H, OCHhd 3), 5.18 (s, 2H, OCH₂Ph), 7.30 -7.51 (m, 5H, Ph), 8.13 (s, 1H, PyH)].

### C. 2-Acetyloxymethyl-5-benzyloxy-4-methoxy-3-methylpyridine

50.0 g (0.19 mol) of 5-benzyloxy-4-methoxy-2,3-dimethylpyridine N-oxide are suspended in 120 ml (1.27 mol) of acetic anhydride. The yellow solution is stirred at reflux overnight and then concentrated to a volume of approximately 20 ml. The residue is taken up in 200 ml of water and extracted with 4×100 ml of ethyl acetate. The organic extracts are washed with saturated sodium chloride solution, dried over sodium sulfate and concentrated. 54.0 g (92.8%) of the title compound are isolated as a brown oil [NMR: δ=2.02 (s, 3H, COCH₃), 2.22 (s, 3H, Me), 3.84 (s, 3H, OCH₃), 5.12 (s, 2H, OCH₂Ph), 5.22 (s, 2H, CH₂OAc), 7.30-7.51 (m, 5H, Ph), 8.21 (s, 1 H, PyH)].

### D. 2-Hydroxymethyl-5-benzyloxy-4-methoxy-3-methylpyridine

54.0 g (0.18 mol) of 2-acetyloxymethyl-5-benzyloxy-4-methoxy-3-methylpyridine are dissolved in methanol. 34.1 ml of 30% (0.18 mol) methanolic sodium methoxide solution are added. Following the addition of a further 100 ml of methanol, the brown suspension is stirred for 30 min. The mixture is concentrated, suspended in ethyl acetate and stirred overnight. The precipitate is filtered off with suction, washed with ethyl acetate and dried in a vacuum drying cabinet at 40° C. 28.5 g (61.4%) of the title compound are isolated as a light-colored solid. m.p. 70-73° C.

### E. (5-Benzyloxy-4-methoxy-3-methylpyridin-2-ylmethyl)(5-chloro-2,6-dimethylpyrimidin-4-yl)amine

28.0 g (0.11 mol) of 2-hydroxymethyl-5-benzyloxy-4-methoxy-3-methylpyridine are suspended in 500 ml of anhydrous dichloromethane and after the suspension has been cooled to 0° C. 8.6 ml (0.12 mol) of thionyl chloride are slowly added dropwise. The solution is cooled to room temperature and stirred for 9 h. It is then poured into 1 l of water, adjusted to a pH of 7.0 using saturated sodium bicarbonate solution, and the organic phase is separated off. The aqueous phase is extracted with 3×200 ml of dichloromethane and the combined organic extracts are dried over sodium sulfate and concentrated. The residue (18.0 g; 64.8 mmol) is dissolved in 200 ml of anhydrous dimethylformamide and added dropwise at 0° C. to a solution of 17.2 g (0.11 mol) of 4-amino-5-chloro-2,6-dimethylpyrimidine and 4.8 g (0.12 mol) of sodium hydride (60% in liquid paraffin) in 400 ml of anhydrous dimethylformamide. The reaction mixture is stirred at 0° C. for 1 h and then poured into 1.2 l of water. The precipitate is filtered off with suction and dried in a vacuum drying cabinet at 40° C. 22.9 g (88.8%) of the title compound are isolated as a light-colored solid. m.p. 113.5-116.0° C.

### F. 6-[(5-Chloro-2,6-dimethylpyrimidin-4-ylamino)methyl]-3-hydroxy-4-methoxy-5-methylpyridine

5.0 g (12.5 mmol) of (5-benzyloxy-4-methoxy-3-methylpyridin-2-ylmethyl)(5-chloro-2,6-dimethyl-pyrimidin-4-yl)amine are suspended in 50 ml of ethanol, and 50 ml of concentrated hydrochloric acid are added. The mixture is first stirred at room temperature for 1 h, then heated to 80° C. and stirred further overnight. The solution is adjusted to a pH of 7.0 using 10N sodium hydroxide solution and the precipitate is filtered off with suction and dried in a vacuum drying cabinet at 40° C. 3.66 g (94.8%) of the title compound are isolated as a pale pink solid. m.p. 220-223.5° C.

### G. (5-Benzyloxypyridin-2-ylmethyl)(5-chloro-2,6-dimethylpyrimidin-4-yl)amine

A solution of 4.7 g (29.9 mmol) of 4-amino-5-chloro-2,6-dimethylpyrimidine in 10 ml of anhydrous N-methylpyrrolidone is added dropwise at room temperature to a suspension of 0.86 g (35.9 mmol) of sodium hydride (60% suspension in liquid paraffin) in 40 ml of anhydrous N-methyipyrrolidone. The mixture is stirred at room temperature for 3 h and then cooled to 0° C. A solution of 7.0 g (29.9 mmol) of 5-benzyoxy-2-chloromethylpyridine in 15 ml of anhydrous N-methylpyrrolidone is slowly added dropwise and the mixture is stirred at 0° C. for 5 h. The mixture is; poured into 200 ml of water and the precipitate is filtered off with suction to give, after drying in a vacuum drying cabinet at 40° C., 4.5 g (43.8%) of the title compound as a beige solid. m.p. 130-131° C.

### H. 6-[(5-Chloro-2,6-dimethylpyrimidin-4-ylamino)methyl]-3-hydroxypyridine hydrobromide

3.2 g (9.0 mmol) of (5-benzyloxypyridin-2-ylmethyl)(5-chloro-2,6-dimethylpyrimidin-4-yl)amine are suspended in 33% hydrogen bromide in acetic acid and the suspension is heated at 45° C. for 3 h. After cooling to room temperature, the solid is filtered off with suction and washed with dichloromethane to give, after drying in a vacuum drying cabinet at 40° C., 2.9 g (93.1%) of the title compound as a white powder. m.p. 208-209° C.

### I. (5-Benzyloxy-6-chloropyridin-2-ylmethyl)-(5chloro-2,6-dimethylpyrimidin-4-yl)amine

2.5 g (15.9 mmol) of 4-amino-5-chloro-2,6-dimethylpyrimidine are added a little at a time to a suspension of 0.7 9 (17.5 mmol) of sodium hydride (60% strength suspension in paraffin oil) in 30 ml of anhydrous dimethylformamide, and the mixture is stirred overnight. A solution of 4.25 g (15.9 mmol) of 2-chloromethyl-5-benzyloxy-6-chloropyridine in 20 ml of anhydrous dimethylformamide is added dropwise. The reaction mixture is cooled to 5° C. and stirred for 2 h. The mixture is then added to 200 ml of water and extracted with ethyl acetate (3×50 ml). The organic extracts are dried over magnesium sulfate and concentrated. The residue is purified by silica gel chromatograpy (petroleum ether/ethyl acetate=1:1). This gives 2.8 g (45%) of the title compound as a white solid.

NMR: δ=2.28 (s, 3H, Me), 2.35 (s, 3H, Me), 4.58 (d, 2H, CH₂), 5.21 (s, 2H, CH₂Ph), 7.22 (d, 1H, PyH), 7.32-7.50 (m, 5H, Ph), 7.62 (d, 1H, PyH), 7.77 (t. 1H, NH).

### J. (5-Benzyloxypyridin-2-ylmethyl)-(5-chloro-2,6-dimethylpyrimidin-4-yl)methylamine

1.1 g (6.2 mmol) of (5-chloro-2,6-dimethylpyrimidin-4-yl)methylamine are added a little at a time to a suspension of 0.25 g (6.5 mmol) of sodium hydride (60% strength suspension in paraffin oil) in 5 ml of anhydrous N-methylpyrrolidone, and the mixture is stirred for 1 h. A solution of 1.5 9 (6.2 mmol) of 2-chloromethyl-5-benzyloxypyridine in 5 ml of anhydrous N-methylpyrrolidone is added dropwise, and the reaction mixture is stirred at room temperature for 2 h. This solution is then added to water and extracted with ethyl acetate (3×50 ml). The organic extracts are dried over magnesium sulfate and concentrated. The residue is purified by silica gel chromatography (toluene/dioxane=4:1). This gives 2.0 g (61%) of the title compound as a yellow oil.

### K. 2-Chloro-6-[(5-chloro-2,6-dimethylpyrimidin-4-ylamino)methyl]-3-hydroxypyridine

2.82 g (6.6 mmol) of (5benzyloxy-6-chloropyridin-2-ylmethyl)-(5-chloro-2,6-dimethylpyrimidin -4yl)amine (from example I) are suspended in ethanol (10 ml) and conc. hydrochloric acid (20 ml) and heated at reflux for 8 h. The clear solution is cooled to room temperature, adjusted to pH=7.0 using saturated sodium bicarbonate solution and stirred at room temperature for 30 min. The precipitate is filtered off with suction and dried in a vacuum drying cabinet at 40° C. 1.65 g (84%) of the title compound are isolated as a white solid.

NMR: δ=2.28 (s, 3H, Me), 2.32 (s, 3H, Me), 4.57 (d, 2H, CH₂), 7.11 (d, 1H, PyH), 7.30 (d, 1H, PyH), 7.68 (t, 1H, NH), 10.5 (s, 1H, OH).

### L. 6-{[(5-Chloro-2,6-dimethylpyrimidin-4-yl)methylamino]methyl}-3-hydroxypyridine

1.6 g (4.4 mmol) of (5-benzyloxypyridin-2-ylmethyl)-(5-chloro-2,6-dimethylpyrimidin-4-yl)methyamine (from example J) are dissolved in methanol (25 ml), and 80 μl of hydrochloric acid (5% strength solution in water), 160 mg of palladium-on-carbon (type 90, Johnson Matthey) and 4.1 ml (43.7 mmol) of cyclohexadiene are added. The mixture is heated at 60° C. for 15 min. After cooling to room temperature and removal of the catalyst by filtration through kieselguhr, the filtrate is added to water and extracted with ethyl acetate (30 ml). The organic phase is washed with saturated sodium bicarbonate solution and extracted with ethyl acetate (3×30 ml). The organic extracts are dried over magnesium sulfate and concentrated. Following silica gel chromatography of the residue (toluene/dioxane=3:2), 909 mg (79%) of the title compound are obtained as a white solid. M.p. 137°-138° C.

### M. 2-Chloro-3-hydroxy-6-methylpyridine

20 g (184 mmol) of 3-hydroxy-6-methylpyridine are dissolved in 80 ml of glacial acetic acid, and a solution of 32 g (240 mmol) of N-chlorosuccinimide in 480 ml of glacial acetic acid is added dropwise. The reaction mixture is stirred at room temperature overnight. A solution of 21 g (110 mmol) of sodium disulfite in 160 ml of water is then added, and the mixture is stirred at room temperature for 1 h and then concentrated. The residue is dissolved in 150 ml of methanol, 120 ml (644 mmol) of sodium methoxide solution (30% strength in methanol) are added and the mixture is stirred at room temperature for 30 min. The solution is then adjusted to pH=7.0 using 2 M hydrochloric acid and concentrated. The residue is poured into water (500 ml) and extracted with ethyl acetate (6×100 ml). The organic extracts are washed with saturated sodium chloride solution, dried over magnesium sulfate and concentrated. The residue (28 g) is chromatographed on silica gel, and the product fractions are collected and concentrated. This gives 14.7 g (56%) of the title compound as a beige solid.

NMR: δ=2.31 (s, 3H, Me), 7.08 (d, 1H, PyH), 7.12 (d, 1H, PyH), 10.3 (s, 1H, OH).

### N. 2-Bromo-3-hydroxy-6-methylpyridine

10 g (91.6 mmol) of 3-hydroxy-2-methylpyridine are dissolved in 180 ml of pyridine and, after cooling to 0° C., 4.7 ml (91.6 mmol) of bromine are slowly added dropwise. The solution is warmed to room temperature and stirred for 2 h. The suspension is then concentrated, the precipitate is filtered off with suction and the mother liquor is extracted with ethyl acetate. The organic extracts are dried over magnesium sulfate and concentrated. The residue (20 g) is purified by silica gel chromatography (petroleum ether/ethyl acetate=4:1). 6.3 g (37%) of the title compound are isolated as a white solid.

NMR: δ=2.31 (s, 3H, Me), 7.10 (d, 1H, PyH), 7.20 (d, 1H, PyH), 10.4 (s, 1H, OH).

### O. 2-Bromo-6-[(5-chloro-2,6-dimethyl-pyrimidin-4-ylamino)-methyl]-3-hydroxy-4-(2,2,2,-trifluoroethoxy)-pyridine

0.59 g (1.1 mmol) of [5-benzyloxy-6-bromo-4-(2,2,2-trifluoro-ethoxy)-pyridin-2-ylmethyl]-(5-chloro-2,6-dimethyl-pyrimidin-4-yl)-amine (from example P) in ethanol (10 ml) are treated with conc. HCl (10 ml) and the reaction mixture is refluxed for 3 h. After cooling to room temperature, the solution is neutralized with 10N NaOH, poured into water and extracted with ethyl acetate (3×20 ml). The combined organic layers are dried with magnesium sulphate and concentrated in vacuo to yield 0.45 g (93%) of the title compound as a beige powder of m.p. 204°-205° C.

### P. [5-Benzyloxy-6-bromo-4-(2,2,2-trifluoro-ethoxy)-pyridin-2-ylmethyl]-(5-chloro-2,6dimethyl-pyrimidin-4-yl)-amine

A solution of 0.75 g (4.8 mmol) of 5-chloro-2,6-dimethyl-pyrimidin-4-ylamine in dry N,N-dimethylformamide (10 ml) is treated with 0.23 g (5.8 mmol) of sodium hydride (60% suspension in paraffine) at 0° C. and the mixture is stirred at this temperature for 15 min. A solution of 1.9 g (4.8 mmol) of 3-benzyloxy-2-bromo-6-chloromethyl-4-(2,2,2-trifluoro-ethoxy)-pyridine in dry N,N-dimethylformamide (5 ml) is added dropwise and the temperature slowly allowed to rise to room temperature. The reaction mixture is stirred at room temperature for 30 min. and then poured into water (150 ml) and extracted with ethyl acetate (3×70 ml). The combined organic layers are dried with magnesium sulphate and concentrated in vacuo. The residue is purified by chromatography on silica gel (petroleum ether/ethyl acetate=3:1) to yield 0.61 g (24%) of the title compound as a white powder of m.p. 97°-98° C.

### Commercial Utility

The excellent activity of compounds of the formula I and their salts against Helicobacter bacteria allows them to be used in human medicine as active principles for treating diseases due to Helicobacter bacteria.

The invention therefore further provides a method of treating mammals, especially humans, who have contracted diseases due to Helicobacter bacteria. The method comprises administering to the individual affected a therapeutically active and pharmacologically tolerated amount of one or more compounds of the formula I and/or their pharmacologically acceptable salts.

The invention further provides the compounds of the formula I and their pharmacologically acceptable salts for use in the treatment of diseases due to Helicobacter bacteria.

The invention likewise embraces the use of compounds of the formula I and their pharmacologically acceptable salts in the preparation of medicaments used for controlling diseases due to Helicobacter bacteria.

The invention additionally provides medicaments for controlling Helicobacter bacteria, comprising one or more compounds of the general formula I and/or their pharmacologically acceptable salts.

Among the Helicobacter strains against which the compounds of the formula I are found effective, mention may be made in particular of the strain Helicobacter pylori, the compounds of the invention being distinguished in particular by high selectivity for Helicobacter microbes.

The medicaments are prepared by conventional methods familiar to the skilled worker. As medicaments, the pharmacologically active compounds of the formula I and their salts (i.e., active principles) are used either as they are or, preferably, in combination with suitable pharmaceutical auxiliaries in the form, for example, of plain tablets, coated tablets, capsules, emulsions, suspensions, gels or solutions, the active principle content being advantageously between 0.1 and 95%.

The choice of suitable auxiliaries for the desired medicament formulations is familiar to the skilled worker on the basis of his or her art knowledge. Besides solvents, gel formers, tableting auxiliaries, and other excipients for the active principle, it is possible, for example, to use antioxidants, dispersants, emulsifiers, defoamers, flavor corrigents, preservatives, solubilizers, colorants or permeation promoters and complexing agents (e.g., cyclodextrins).

The active principles may be administered, for example, parenterally (e.g., intravenously) or, in particular, orally.

In human medicine, in general, the active principles are administered in a daily dose of from about 0.1 to 50, preferably from 1 to 30, mg/kg of body weight, where appropriate in the form of two or more, preferably 2 to 3, individual doses, in particular a single dose daily, in order to achieve the desired result.

The compounds of the invention may also be administered in a fixed or free combination together with a substance which neutralizes gastric acid and/or inhibits gastric acid secretion and/or with a substance suitable for conventional control of Helicobacter pylori.

Examples of gastric acid neutralizers include sodium bicarbonate or other antacids (such as aluminum hydroxide, magnesium aluminate or magaldrate). Examples of gastric acid secretion inhibitors that may be mentioned include H₂ blockers (e.g., cimetidine, ranitidine), H⁺/K⁺ ATPase inhibitors (e.g., lansoprazole, omeprazole, esomeprazole, rabeprazole or, in particular, pantoprazole) and what are known as reversible H⁺/K⁺ ATPase inhibitors (compounds as disclosed, for example, in international patent applications WO 00/11000, WO 00/10999, WO 99/55706, WO 99/55705 or WO 98/37080, and structurally similar compounds).

As substances suitable for the conventional control of Helicobacter pylori, mention may be made in particular of antimicrobial substances such as, for example, penicillin G, gentamycin, erythromycin, clarithromycin, azithromycin, nitrofurazone, tinidazole, nitrofurantoin, furazolidon, ampicillin, cefaclor, cefadroxil, cefalexin, cefpodoxime proxetil, cefradine, ceftazidime, ceftriaxone, cefuroxime, ciprofloxacin, clindamycin, doxycycline, ecabet, gatifloxacin, imipenem, meropenem, mezlocillin, minocycline, moxifloxacin, norfloxacin, ofloxacin, oxetacaine, paromomycin, pefloxacin, rebamipide, rifampicin, rifaximin, roxatidine, tetracycline, tiabendazole, trovafloxacin, ritipenem, ecabapide, nitazoxanide, sanfetrinem, sitafloxacin, trospectomycin, metronidazole or amoxycillin, or else bismuth salts such as bismuth citrate, for example.

### Biological Investigations

### Agar Dilution Test (Determination of the Inhibition of Growth In Vitro on Agar Plates)

The compounds of the formula I were investigated for their activity against Helicobacter pylori in accordance with the methodology described by Tomoyuki Iwahi et al. (Antimicrobial Agents and Chemotherapy, 1991, 490-496) using Columbia agar (Oxoid) over a growth period of 4 days. The compounds investigated gave the approximate MIC₅₀ values set out in table A below (the numbers of the compounds indicated correspond to the numbers of examples in the description).

TABLE A

| Compound | |
|---|---|
| No. | approx. MIC₅₀ (mg/l) |
| 1 | 0.1 |
| 2 | 0.1 |
| 3 | 0.1 |
| 4 | 0.1 |

### Determination of the Inhibition of Growth In Vitro in Liquid Culture

The principle of the technique is based on the detection of the multiplication of, for example, Helicobacter pylori in liquid culture using BHI/6% FCS medium. The method ensures linear fluorescence increase in the range from 3×10⁶ to 3×10⁸ cells.

The bacterial culture was distributed with an initial density of 1-3×10⁶ microbes/ml in a 96-well MTP in 100 μaliquots. The test substances in a concentration of 10⁹ to 10⁵ mol/l in a final concentration of 1% DMSO were added to these minicultures. These MTPs were then incubated under microaerobic conditions (Anaerokult, Merck) and with shaking at 37° C. for 24 hours. Following the 24-hour incubation, the minicultures were transferred to filter MTPs and washed twice with isotonic buffer (filtered off with suction, taken up, shaken) and finally were taken up in double-distilled water and shaken, and an aliquot was transferred to a new MTP. This aliquot was admixed with the fluorescent dye NanoOrange (Molecular Probes) in accordance with the manufacturer's instructions. Development of protein detection took place at 90° C. in a pressure-secured sandwich technique. After the plates had cooled, the fluorescence was measured on a plate reader at 549 nm. These data were used to construct concentration/effect curves from which the parameters of the substances, the IC₅₀ values, were determined. This calculation was made using origin, sigmoidal curve adaptation by means of the ‘logistic’ algorithm. The compounds investigated in this technique gave the IC₅₀ values (the numbers of the compounds indicated correspond to the numbers of examples in the description) set out in table B below.

TABLE B

| Compound | |
|---|---|
| No. | IC₅₀ (μmol/l) |
| 1 | 0.012 |
| 2 | 0.064 |
| 3 | 0.035 |
| 4 | 0.04 |

### Determination of the Helicobacter pylori Eradication Rate In Vivo

Experimental Setup:

Gerbils were infected on days 1, 3, and 5 with a suspension containing 10⁸-10⁹ Helicobacter pylori bacteria per animal. Following infection, the gerbils had a recovery phase of 4 weeks within which the bacteria were able to colonize the stomach. Beginning on day 36, the gerbils were treated on four successive days—three times daily at 07.30, 11.30, and 15.00 hours—with a placebo or the test substance, using a tube. Four weeks after the last treatment, the gerbils were sacrificed using CO₂. A tissue sample of the antrum was introduced into the urease test solution and incubated at 37° C. for 24 hours. Changes in color of the solution from yellow to violet, which resulted from the increase in pH caused by the formation of NH₃ from the urease, were detected. The eradication rate was calculated as the percentage of animals whose stomach tissue sample gave a negative urease test.

Conditions Under which the Animals were Kept:

Groups of 5-10 gerbils per cage (type IV Macrolon cage) were kept at an ambient temperature of 23±2° C. and a relative humidity of 50±10%. They were fed ad libitum with NAFAG feed No. 9439 for rats and mice (NAFAG AG, CH-2900, Gossau, Switzerland) and had free access to mains water during the experiment.

Substances and dosages:

| | |
|---|---|
| Dissolution proportion of the substance: | 4% methylcellulose in water |
| Volume administered: | 10 ml/kg |
| Form of administration: | tube |
| Frequency of administration: | 3 × daily |
| Duration of therapy: | 4 days |

The substances administered are referenced in table C below using numbers which correspond to the numbers of the compounds in the examples.

TABLE C

| | Dose administered | Eradication rate |
|---|---|---|
| Compound No. | in mg/kg | in % |
| 3 | 50 | 100 |

1. A compound of the formula I, 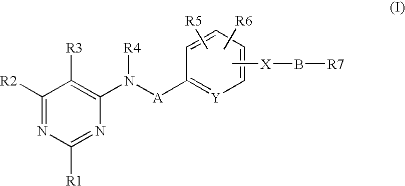
in which
R1 is hydrogen, 1-4C-alkyl or halogen,
R2 is hydrogen, 1-4C-alkyl or halogen,
R3 is hydrogen, 1-4C-alkyl or halogen,
R4 is hydrogen or 1-4C-alkyl,
R5 is hydrogen, hydroxyl, 1-4C-alkyl, 1-4C-alkoxy, wholly or predominantly fluorine-substituted 1-4C-alkoxy, trifluoromethyl or halogen,
R6 is hydrogen, hydroxyl, 1-4C-alkyl, 1-4C-alkoxy, wholly or predominantly fluorine-substituted 1-4C-alkoxy, or halogen,
R7 is a cyclic or bicyclic radical which is substituted by nitro and R8 and R9 and is selected from the group consisting of imidazole, imidazopyridazine and imidazopyridine,
A is 1-7C-alkylene,
B is a bond or 1-7C-alkylene,
X is O (oxygen), N-1-4C-alkyl, NH or S(O)ₙ and
Y is N,
where
R8 is hydrogen, 1-4C-alkyl, halogen, nitro, hydroxy-1-4C-alkyl or 1-4C-alkylcarbonyloxy-1 -4C-alkyl,
R9 is hydrogen, 1-4C-alkyl or nitro, and
n is0, 1 or 2,
and salts thereof,
where the following is excluded
a compound of the formula I in which
A is methylene or methylene substituted by one or two 1-4C-alkyl
R5 and R6 are hydrogen, 1-4C-alkyl, 1-4-C-alkoxy or halogen
X is attached to the 4 position of the pyridine ring
a) B is 2-7C-alkylene X is N-14C-alkyl
b) B is 1 -7C-alkylene X is O (oxygen)
c) B is 1 -7C-alkylene X is S
and salts thereof.

2. A compound of the formula I as claimed in claim 1, wherein
R1 is hydrogen, 1-4C-alkyl or halogen,
R2 is hydrogen, 1-4C-alkyl or halogen,
R3 is hydrogen or halogen,
R4 is hydrogen or 1-4C-alkyl,
R5 is hydrogen, hydroxyl, 1-4C-alkyl, 1-4C-alkoxy, wholly or predominantly fluorine-substituted 1-4C-alkoxy, trifluoromethyl or halogen,
R6 is hydrogen, 1-4C-alkyl, 1-4C-alkoxy or halogen,
R7 is a cyclic or bicyclic radical which is substituted by nitro and R8 and R9 and is selected from the group consisting of imidazole and imidazopyridazine,
A is methylene,
B is a bond or 1-4C-alkylene,
X is O (oxygen), NH or S(O)ₙ and
Y is N
where
R8 is hydrogen,
R9 is hydrogen,
n is 0,
and salts thereof,
where the following is excluded
a compound of the formula I, in which
R5 is hydrogen, 1-4C-alkyl, 1-4C-alkoxy or halogen
X is attached to the 4 position of the pyridine ring
B is 1-4C-alkylene
a) X is O (oxygen)
b) X is S and salts thereof.

3. A compound of the formula I as claimed in claim 1, wherein
R1 is hydrogen or 1-4C-alkyl,
R2 is hydrogen or 1-4C-alkyl,
R3 is halogen,
R4 is hydrogen or 1-4C-alkyl,
R5 is hydrogen, 1-4C-alkyl, 1-4C-alkoxy, wholly or predominantly fluorine-substituted 1-4C-alkoxy or halogen,
R6 is hydrogen, 1-4C-alkyl, 1-4C-alkoxy or halogen,
R7 is a cyclic or bicyclic radical which is substituted by nitro and R8 and R9 and is selected from the group consisting of imidazole and imidazopyridazine,
A is methylene,
B is a bond or 1-4C-alkylene,
X is O (oxygen) and
Y is N
where
R8 is hydrogen or 1-4C-alkyl,
R9 is hydrogen,
and salts thereof,
where the following is excluded
a compound of the formula I, in which
R5 is hydrogen, 1-4C-alkyl, 1-4C-alkoxy or halogen
X is attached to the 4 position of the pyridine ring
B is 1-4C-alkylene
X is O (oxygen) and salts thereof.

4. A compound of the formula I as claimed in claim 1, wherein
R1 is hydrogen or methyl,
R2 is hydrogen or methyl,
R3 is hydrogen or chlorine,
R4 is hydrogen or methyl,
R5 is hydrogen, 1-4C-alkyl, 1-4C-alkoxy, wholly or predominantly fluorine-substituted 1-4C-alkoxy, trifluoromethyl or halogen,
R6 is hydrogen, 1-4C-alkyl, 1-4C-alkoxy or halogen,
R7 is a 3-nitroimidazo[1,2-b]pyridazin-6-yl radical or a 2methyl-5-nitroimidazol-1-yl radical,
A is methylene,
B is a bond or 1-2C-alkylene,
X is O (oxygen), NH or S, and
Y is N,
and salts thereof,
where the following is excluded
a compound of the formula I, in which
R5 is hydrogen, 1-4C-alkyl, 1 -4C-alkoxy or halogen
X is attached to the 4 position of the pyridine ring
B is 1-2C-alkylene
a) X is O (oxygen)
b) X is S and salts thereof.

5. A compound of the formula I as claimed in claim 1, 2, 3 or 4, wherein B is a bond and R7 is a 3-nitroimidazo[1,2]pyridazin-6-yl radical.

6. A compound of the formula I as claimed in claim 1, 2, 3 or 4, wherein B is an ethylene radical and R7 is a 2-methyl-5-nitroimidazol-1-yl radical.

7. A compound of the formula I as claimed in claim 1, 2, 3 or 4, wherein B is a bond, R7 is a 3-nitroimidazo[1,2-b]pyridazin-6-yl radical and X is O (oxygen).

8. A compound of the formula I as claimed in claim 1, 2, 3 or 4, wherein B is an ethylene radical, R7 is a 2-methyl-5-nitroimidazol-1-yl radical and X is O (oxygen).

9. A compound as claimed in claim 1, characterized by the formula I*, 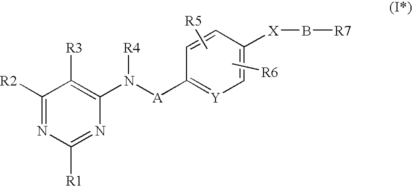
in which
R1 is hydrogen or methyl,
R2 is hydrogen or methyl,
R3 is hydrogen or chlorine,
R4 is hydrogen or methyl,
R5 is hydrogen, hydroxyl, methyl, methoxy, ethoxy, cyclopropylmethoxy, isobutoxy, trifluoromethoxy, difluoromethoxy, trifluoromethyl or chlorine,
R6 is hydrogen, methyl, methoxy or chlorine,
R7 is a 3-nitroimidazo[1,2-b]pyridazin-6-yl radical,
A is methylene,
B is a bond,
X is O (oxygen), NH or S, and
Y is N,
and salts thereof.

10. A compound as claimed in claim 1, characterized by the formula I* as defined in claim 9, in which
R1 is hydrogen or methyl,
R2 is hydrogen or methyl,
R3 is hydrogen or chlorine,
R4 is hydrogen or methyl,
R5 is hydrogen, hydroxyl, methyl, methoxy, ethoxy, cyclopropylmethoxy, isobutoxy, trifluoromethoxy, difluoromethoxy, trifluoromethyl or chlorine,
R6 is hydrogen, methyl, methoxy or chlorine,
R7 is a 2-methyl-5-nitroimidazo1-yl radical,
A is methylene,
B is ethylene,
X is O (oxygen), NH or S, and
Y is N,
and salts thereof.

11. A compound as claimed in claim 1, characterized by the formula I* as defined in claim 9, in which
R1 is 1-4C-alkyl,
R2 is 1-4C-alkyl,
R3 is halogen,
R4 is hydrogen or 1-4C-alkyl,
R5 is hydrogen, 1-4C-alkyl, 1-4C-alkoxy, wholly or predominantly fluorine-substituted 1-4C-alkoxy or halogen,
R6 is hydrogen, 1-4C-alkyl, 1-4C-alkoxy or halogen,
R7 is a 2-methyl-5-nitroimidazo-1-yl radical or a 3-nitroimidazo[1,2-b]pyridazin-6-yl radical,
A is methylene,
B is a bond or ethylene,
X is O (oxygen) and
Y is N, and salts thereof.

12. A compound as claimed in claim 1, characterized by the formula I* as defined in claim 9, in which
R1 is methyl,
R2 is methyl,
R3 is chlorine,
R4 is hydrogen or methyl,
R5 is hydrogen, methyl, methoxy, trifluoroethoxy or chlorine,
R6 is hydrogen, methyl, methoxy or bromine,
R7 is a 2-methyl-5nitroimidazo-1-yl radical or a 3-nitroimidazo[1,2-b]pyridazin-6-yl radical,
A is methylene,
B is a bond or ethylene,
X is O (oxygen) and
Y is N,
and salts thereof.

13. A medicament comprising compounds of the formula I as claimed in claim 1 and/or their pharmacologically acceptable salts together with customary auxiliaries.

14. The use of compounds of the formula I as claimed in claim 1 and/or their pharmacologically acceptable salts in the control of Helicobacter bacteria.

15. The use of compounds of the formula I as claimed in claim 1 and their pharmacologically acceptable salts for preparing medicaments for controlling Helicobacter bacteria.
